(19) United States
(12) Patent Application Publication
(10) Pub. No.: US 2011/0213171 A1
(43) Pub. Date: Sep. 1, 2011

(54) THE WAY OF PREPARING VEGETABLE OILS (TRI-GLICERIDES) FOR POWERING JET ENGINES.

(76) Inventor: Kazimierz Malinowski, Mirkow (PL)

(21) Appl. No.: 13/129,356

(22) PCT Filed: Dec. 27, 2008
(86) PCT No.: PCT/PL2008/000098

§ 371 Date: May 13, 2011

(30) Foreign Application Priority Data
Dec. 21, 2008 (PL) P.386879

(51) Int. Cl. C07C 59/235

(52) U.S. Cl. 554/122

(57) ABSTRACT

The way of preparing vegetable oils (tri-glicerides) for powering jet engines, where vegetable oils (tri-glicerides) are made of 18 carbon atoms C 18, and molecule of tri-gliceride is built from carbon, hydrogen and oxygen, and in addition tri-gliceride is made of three carbon atoms joined to oneself with single bonds, and every carbon atom is combined moreover with single bonds with a hydrogen atom on one side and with atoms of oxygen on the other, and in addition it's characteristic, that ‘outermost’, four-value carbon atoms are connected with hydrogen atoms, while centre, three-value carbon atom with one hydrogen atom, moreover every carbon atom, through oxygen atoms, is connected with next four-value carbon atom, making the base of the tri-gliceride twig, and four-value carbon atoms are connected through single bonds with the oxygen atom (and backwards with carbon atoms) through double connection with different atoms of oxygen and through the single bond with so-called radical, which is a simple hydrocarbon chain depends on the fact, that for powering the jet engine the vegetable oil should be held in the scope of temperatures in an optimal way from −14° C. to +120° C., and before injecting directly into the jet engine it should be heated to the range of temperatures in an optimal way from +285° C. to +350° C.

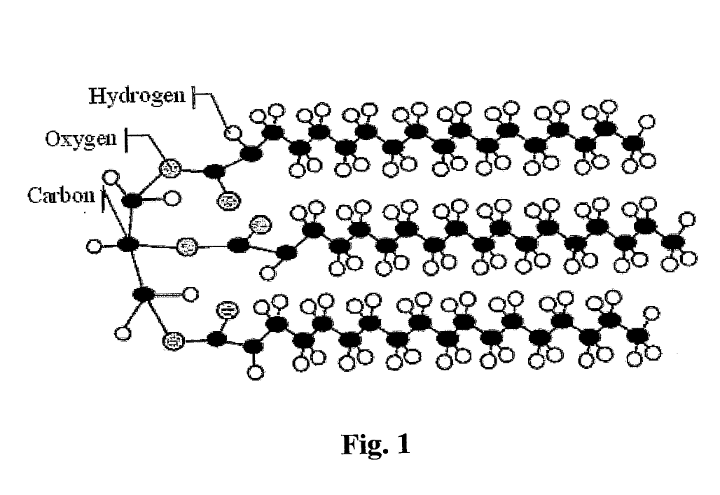

The way of preparing vegetable oils is an object (subject) of the invention (tri-glicerides) for powering jet engines.

Attempts to start the jet engine on the vegetable oil (tri-gliceride) are well-known, every attempt ended in failure at the preliminary stage of launching vegetable oils (tri-glicerides) with the help of the torch. It resulted from the fact that nobody so far had mastered the technology of treating vegetable oils (tri-glicerides). However I noticed, that vegetable oils (tri-glicerides) aren't evaporating, nor they also undergo ignition to the temperature of +285° C. Therefore I reached a conclusion that it was possible to heat them to the +285° C. temperature and it will cause neither the phenomenon of evaporationg nor of ignition of vegetable oils (tri-glicerides) heated to this temperature. Thanks to this phenomenon I stated that this fuel was perfect for powering jet engines, because at holding the temperature from −14° C. to +120° C. in the container vegetable oil (tri-glicerides) won't block the fuel filter and vegetable oils are blocking fuel filters at the temperature of −14° C., however they are crystallizing in the temperature of −20° C. However once more heated right before pouring into the jet engine in the temperature from +285° C. to +350° C. they have identical properties (the viscosity and the flashpoint) as classical kerosenes for powering jet engines. And so vegetable oils (tri-glicerides) have the advantage over classical kerosenes used so far for powering jet engines, that they are non-inflammable and aren't evaporating to the +285° C. temperature, which is a desired phenomenon, because in the case of the disaster they won't cause ignition of vegetable oils (tri-glicerides), because they are evaporating and they are ignited above the +285° C. temperature. Moreover thanks to the fact that it is possible to heat it in containers in the scope of temperatures in an optimal way from −14° C. to +120° C., it is impossible to ignite vegetable oils (tri-glicerides), because their evaporating and ignition temperature is higher than +285° C. Therefore they should be heated in the containers of aeroplanes in the optimal scope of temperatures from −14° C. to +120° C., and thanks to that the jet engine can work in lower temperatures than powered with typical JET 1 or JET 2 kerosenes. It means that jet aeroplane powered with vegetable oils (tri-glicerides) will fly irrespective of how the surrounding temperature is low all the way to the height where essential oxygen is still appearing for the process of igniting vegetable oils (tri-glicerides), that is arounf 30 000 m above sea level., that is above twice higher than the jet engine powered with the ethanol.

This kind of fuel will have fuels double applying: civilian and military.

Civilian: very much firmly a safety will improve during the start and landing, as well as during the flight and it will reduce operating costs of powered jet aircraft with vegetable oils (tri-glicerideami), because at maximum heights there is such considerable thinning of air, that there are minimum resistances of the flight and therefore the sound barrier is moved, which means, that the aeroplane can fly with the speed above 1000 km/h without crossing the sound barrier.

Military: engines of jet aircraft powered with vegetable oils (tri-glicerides) will be working at maximum heights, where the minimum amount of oxygen essential to combustion process is appearing, i.e. about 30 000 m. above sea level and fly with the speed repeatedly higher than 1000 km/h, in addition not to cross the sound barrier (air considerably thinned down), which is giving the gigantic advantage of the height over classical jet aircrafts.

In the present state of technique flying above the height of the 16 500 m above sea level was impossible, because it threatened with blocking of fuel filters, however my invention allow to fly above this height, because fuel in containers will be heated with the heat from the working jet engine, and so at the same time it will be fuel and the coolant.

The invention consists in preparing vegetable oils (tri-glicerides) for powering jet engines where vegetable oils (tri-glicerides) are made of 18 carbon atoms C 18, and molecule of tri-gliceride is built from carbon, hydrogen and oxygen, and in addition tri-gliceride is made of three carbon atoms joined to oneself with single bonds, and every carbon atom is combined moreover with single bonds with a hydrogen atom on one side and with atoms of oxygen on the other, and in addition it's characteristic, that “outermost”, four-value carbon atoms are connected with hydrogen atoms, while centre, three-value carbon atom with one hydrogen atom, moreover every carbon atom, through oxygen atoms, is connected with next four-value carbon atom, making the base of the tri-gliceride twig, and four-value carbon atoms are connected through single bonds with the oxygen atom (and backwards with carbon atoms) through double connection with different atoms of oxygen and through the single bond with so-called radical, which is a simple hydrocarbon chain in this way, that vegetable oil intended for powering the jet engine should be held in the range of temperatures in an optimal way from −14° C. to +120° C., and before injecting directly into the jet engine it should be heated to the range of temperatures in an optimal way from +285° C. to +350° C.

Fundamental merits of the invention are that vegetable oils (tri-glicerides) are ecological fuel, biodegradable, very difficult-combustible in the surrounding temperature (a wick is needed), which is safe in the scope of fire-fighting and anti-explosive, very much desired for economical accounts.

The way according to the invention was presented in the example of making, where the vegetable oil(tri-gliceride) was used, made of rape oil built from 18 carbon atoms C 18, and molecule of tri-gliceride is built from carbon, hydrogen and oxygen, and in addition tri-gliceride is made of three carbon atoms joined to oneself with single bonds, and every carbon atom is combined moreover with single bonds with a hydrogen atom on one side and with atoms of oxygen on the other, and in addition it's characteristic, that “outermost”, four-value carbon atoms are connected with hydrogen atoms, while centre, three-value carbon atom with one hydrogen atom, moreover every carbon atom, through oxygen atoms, is connected with next four-value carbon atom, making the base of the tri-gliceride twig, and four-value carbon atoms are connected through single bonds with the oxygen atom (and backwards with carbon atoms) through double connection with different atoms of oxygen and through the single bond with so-called radical, which is a simple hydrocarbon chain, of which the schema is presented in the drawing below and this rape oil is intended for powering the jet engine was held in the container in the +1° C. temperature, and before injecting directly into the jet engine it was heated to the +285° C. temperature.

1. The way of preparing vegetable oils (tri-glicerides) for powering jet engines, where vegetable oils(tri-glicerides) are made of 18 carbon atoms C 18, and molecule of tri-gliceride is built from carbon, hydrogen and oxygen, and in addition tri-gliceride is made of three carbon atoms joined to oneself with single bonds, and every carbon atom is combined moreover with single bonds with a hydrogen atom on one side and with atoms of oxygen on the other, and in addition it's characteristic, that “outermost”, four-value carbon atoms are connected with hydrogen atoms, while centre, three-value carbon atom with one hydrogen atom, moreover every carbon atom, through oxygen atoms, is connected with next four-value carbon atom, making the base of the tri-gliceride twig, and four-value carbon atoms are connected through single bonds with the oxygen atom (and backwards with carbon atoms) through double connection with different atoms of oxygen and through the single bond with so-called radical, which is a simple hydrocarbon chain characteristic with the fact that vegetable oil intended for powering the jet engine should be held in the range of temperatures in an optimal way from -14° C. to +120° C., and before injecting directly into the jet engine it should be heated it in the range of temperatures in an optimal way from +285° C. to +350° C.
